(19) United States
(12) Patent Application Publication
(10) Pub. No.: US 2003/0060503 A1
(43) Pub. Date: Mar. 27, 2003

(54) Nutritional supplements for mature pets

(75) Inventor: Nathan D. Hamilton, Palo Alto, CA (US)

Correspondence Address: SIERRA PATENT GROUP, LTD., P O BOX 6149, STATELINE, NV, 89449, US

(73) Assignee: Juvenon, Inc.

(21) Appl. No.: 10/218,689

(22) Filed: Aug. 12, 2002

Related U.S. Application Data
(63) Continuation-in-part of application No. 09/770,535, filed on Jan. 25, 2001, now abandoned.
(60) Provisional application No. 60/178,073, filed on Jan. 25, 2000.
(60) Provisional application No. 60/223,586, filed on Aug. 7, 2000.

(51) Int. Cl. A61K 38/43; A61K 31/385; A61K 31/198

(52) U.S. Cl. 514/440; 424/94.1; 514/561; 514/546

(57) ABSTRACT

Disclosed herein are compositions to meet the needs of aged pets and other animals. A pet food formulated for senior pets provides α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day. A pet treat for senior pets provides α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day. A pet supplement for mature pets offers α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day.

### CROSS RELATED

This application is a continuation in part of U.S. application Ser. No. 09/770,535, filed Jan. 25, 2001, which claims the benefit of U.S. Provisional Application No. 60/178,073, filed Jan. 25, 2000, and U.S. Provisional Application No. 60/223,586, filed Aug. 7, 2000.

### TECHNICAL FIELD

The present invention is generally directed to pet food and dietary supplements. More specifically, the present invention relates to the addition of the combination of lipoic acid and carnitine to these compositions. Optional additional ingredients are coenzyme Q and creatine.

### BACKGROUND OF INVENTION

Many pet foods contain nutrition for a specific stage of the pet's life. Stages of a pet's life are broken down as follows: kitten or puppy is up to 1 year, adult cat or dog is one to six years, and a senior cat or dog is over six years old. However, different animals age at different rates. Cats are often considered older or senior at seven to eight years of age and geriatric or very old at 10 to 12 years. Dogs often are considered older between 7.5 and 13.5 years of age. Dogs often are considered older when they reach half of their life expectancy, which corresponds to about five years for larger dogs and seven years for smaller dogs.

Today, pets are living markedly longer because of improved treatments for infections and parasites, improved diagnostics, and better medical technology. Improved nutrition also has played a role, particularly the life-stage concept that recognizes different nutritional needs at different ages.

Nutrition is particularly important in aging pets and in managing the risk factors of cancer, heart/cardiac, kidney and liver disease which are prominent causes of non-accidental death in dogs and cats. In addition, older pets become less active and have reduced lean body mass. For these two reasons, pets require less energy from their food. Aging pets also have a reduced immune response and glucose tolerance.

The goals of pet foods for older animals have been stated as maintaining optimal nutrition, managing risk factors, managing diseases, and improving quality and longevity of life. So far, that has meant reducing protein, fat, energy sources, phosphorus and sodium and increasing water and fiber. However, very old dogs (greater than 12 years) may require somewhat more fat and energy sources.

An example of a formulation for older dogs is the Science Diet® Canine Senior® product that provides fewer calories, more fiber and lower phosphorus. The dry formula has 350 kcal/cup. It has the following nutrient contents per 100 kcal: protein 4.8 g, fat 2.8 g, carbohydrate 16.5 g, crude fiber 0.8 g, calcium 155 mg, phosphorus 144 mg, sodium 45 mg, potassium 163 mg, chloride 141 mg and magnesium 30 mg. It also contains the following vitamins: choline chloride, vitamin A, vitamin D3, vitamin E, niacin, thiamine, calcium pantothenate, pyridoxine hydrochloride, riboflavin, folic acid, biotin and vitamin B 12.

A canned turkey Canine Senior® formula provides about 393 kcal per 418-gram can. It has the following nutrient contents per 100 kcal: protein 4.9 g, fat 3.2 g, carbohydrate 15.8 g, crude fiber 0.5 g, calcium 159 mg, phosphorus 138 mg, sodium 43 mg, potassium 181 mg, chloride 149 mg and magnesium 23 mg. It also contains the following vitamins: D-activated animal sterol, vitamin E, niacin, thiamine, calcium pantothenate, pyridoxine hydrochloride, riboflavin, folic acid, biotin and vitamin B12.

The Science Diet Feline Senior™ canned fish formula offers lower energy and higher fiber than pet food for younger cats. It provides 150 kcal per 156-gram can. It has the following nutrient contents per 100 kcal: protein 9.5 g, fat 5.8 g, carbohydrate 5.3 g, crude fiber 1.1 g, calcium 219 mg, phosphorus 177 mg, sodium 115 mg, potassium 198 mg, chloride 177 mg, magnesium 17 mg, and taurine 146 mg. It also contains the following vitamins: vitamin A, D-activated animal sterol, vitamin E, niacin, thiamine, calcium pantothenate, pyridoxine hydrochloride, riboflavin, folic acid, biotin and vitamin B12.

Recent research has suggested that taking sufficient quantities of certain substances rejuvenates aged mitochondria, the failing powerhouses of cell metabolism. Numerous lines of evidence suggest that the organelles of cellular respiration, the mitochondria, degenerate with cellular aging (Shigenaga et al. PNAS 91: 10771, 1994). Unfortunately, the study of mitochondrial aging has been hampered because mitochondria isolated from older cells and host animals are fragile and heterogeneous. Hence the interpretation of any results is suspect as about half the mitochondria lyse during isolation. Recently a new method was developed for studying mitochondria in hepatocytes from old animals that avoids this problem (Hagen et al. PNAS 94, 3064-3069, 1997). Mitochondria from older animals are not only more fragile, but have about half the level of cardiolipin, a key lipid unique to mitochondria, without which they can not maintain a high membrane potential. Furthermore, Hagen et al. showed that in hepatocytes from older animals, the mitochondria are lower in membrane potential and leak more toxic oxidants.

Carnitine and carnitine derivatives have been used as metabolites in animal husbandry and for human diet and therapy. U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); U.S. Pat. No. 5,240,961 (Method of treating reduced insulin-like growth factor and bone loss associated with aging); etc.

Similarly, mitochondrially active antioxidants including vitamins (especially C, E, B and D), glutathione, N-acetyl cysteine, lipoic acid, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); U.S. Pat. No. 5,326,699 (Serum-free medium for culturing animal cells); etc.

Coenzyme Q or ubiquinone has been used as a medicine or food supplement. For example, uses of ubiquinone include U.S. Pat. No. 6,090,414 (Method and composition to reduce cancer incidence); U.S. Pat. No. 6,086,190 (Food supplements); U.S. Pat. No. 6,080,788 (Composition for Improvement of cellular nutrition and mitochondrial energetics); U.S. Pat. No. 6,080,388 (Cosmetic and dermatological sunscreen formulations); U.S. Pat. No. 6,063,432 (Fruit health bar formulation); U.S. Pat. No. 6,048,846 (compositions used in human treatment); U.S. Pat. No. 6,048,566 (Non-alcoholic beverage and process of making), etc.

Creatine has enjoyed increasing use as a nutritional additive by athletes. Other uses of creatine are discussed in U.S. Pat. No. 6,093,746 (Therapeutic agents for asthma); U.S. Pat. No. 6,071,962 (Oxa acids and related compounds for treating skin conditions); U.S. Pat. No. 6,060,512 (Method of using hydroxycarboxylic acids or related compounds for treating skin changes associated with intrinsic and extrinsic aging); U.S. Pat. No. 6,013,290 (Assemblage of nutrient beverages and regimen for enhancing convenience, instruction and compliance with exercise supplementation); U.S. Pat. No. 6,008,253 (Use of 3-guanidino propionic acid to increase endurance, stamina and exercise capacity); U.S. Pat. No. 6,008,252 (Method for increasing muscle mass); etc.

What is needed is an improved nutritional pet food which truly is formulated to meet the needs of older pets. A survey of pet food Web sites uncovered no formula providing carnitine or lipoic acid. Such a pet food would also provide the latest in anti-aging compounds that have been shown to increase energy and stamina, with fewer calories.

### SUMMARY OF INVENTION

It is an object of the present invention to improve pet diets, preferably in pets with deficient mitochondrial metabolism. It is a further object to provide a combination of an effective amount of a suitable antioxidant and an effective amount of a carnitine in a wide variety of foods and food supplements. It is a further object of the present invention to improve the diet of mature dogs, cats, horses, fish, birds and other animals.

A pet food formulated for senior pets provides α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day. In a preferred embodiment, the carnitine is acetyl-L-carnitine. In a preferred embodiment, the α-lipoic acid is R-α-lipoic acid. In yet another embodiment, the coenzyme Q is coenzyme Q10.

In another embodiment, there is provided a pet treat for senior pets, the treat formulated so as to provide α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day. In a preferred embodiment, the carnitine is acetyl-L-carnitine. In a preferred embodiment, the α-lipoic acid is R-α-lipoic acid. In yet another embodiment, the coenzyme Q is coenzyme Q10.

In another embodiment, there is a pet supplement for mature pets, the supplement offering α-lipoic acid at about 0.15 to 50 mg/kg body weight/day, carnitine at about 0.5 to 100 mg/kg/day, and optionally coenzyme Q at about 0.01 mg/kg/day and/or creatine at about 15 mg to about 1 g/kg/day. In a preferred embodiment, the carnitine is acetyl-L-carnitine. In a preferred embodiment, the α-lipoic acid is R-α-lipoic acid. In yet another embodiment, the coenzyme Q is coenzyme Q10.

### DETAILED DESCRIPTION

Pet foods lack four important ingredients: carnitine, lipoic acid, coenzyme Q and creatine. These constituents are essential to discourage aging and provide more energy to older animals and others with unhealthy mitochondria. Recent research has shown precisely how these compounds work to promote healthy mitochondria, which are the energy powerhouses of the cells. Mitochondria are responsible for the production of ATP and are present in relatively high numbers in essentially all cells of the body. The mitochondrial electron transport system consumes approximately 85% of the oxygen utilized by a cell. Cellular energy deficits caused by declines in mitochondrial function can impair normal cellular activities and compromise the cell's ability to adapt to various physiological stresses, a major factor in aging. Because of this high oxygen use, the mitochondria also have the highest production of oxidants.

Oxidants damage mitochondria in three important ways. Oxidants damage DNA, lipids and protein. The intra-mitochondrial DNA (mtDNA) have levels of oxidative damage which are at least 10-fold higher than those of nuclear DNA, which correlates with the 17-fold higher evolutionary mutation rate in mtDNA compared with nuclear DNA. mtDNA oxidation accumulates as a function of age, which has been shown in several species, including humans. This may lead to dysfunctional mitochondria. Mitochondrial protein damage is also age-related and may decrease energy production and increase oxidant production. Oxidative damage to mitochondrial lipids contributes to the decreasing fluidity of cell membranes with age. The lipid cardiolipin is a major component of the mitochondrial membrane and facilitates the activities of key mitochondrial inner membrane enzymes. The aged, damaged mitochondrial membrane cannot contain the oxidants, nor can it maintain as high a polarity as the younger membrane.

Fatty acid oxidation is an important energy source for many tissues. The activity of carnitine-acetyl-carnitine exchange across the inner mitochondrial membrane is of great importance. The activity of this exchange reaction is decreased significantly with age, which may be due to a lower intra-mitochondrial pool of carnitine. L-carnitine or acyl-L-carnitine (ALC) has been shown to slow or reverse this age-related dysfunction. It also can reverse the age-related decrease in cardiolipin, age-associated decrease in mtDNA transcription, and decreased membrane potential. By itself, L-carnitine or ALC cannot correct the problem of excess oxidants. In fact, it was recently reported that carnitine supplementation increased oxidant production by 30% and decreased cell antioxidants markedly. Thus, ALC administration in older individuals may contribute to greater oxidative stress.

For the aged mitochondrial engines to run on all cylinders, both carnitine and lipoic acid are essential. Lipoic acid is an antioxidant. And R-α-lipoic acid is a mitochondrial enzyme which can help reverse the decline in metabolism seen with age. R-α-lipoic acid supplementation has been shown to 1) reverse the age-related decrease in oxygen consumption, 2) restore the age-related decline in mitochondrial membrane potential, 3) triple the ambulatory activity of aged rats, 4) significantly lower the age-related increase in oxidants, and 5) restore glutathione and ascorbic acid levels to youthful levels.

Clearly, both carnitine and lipoic acid contribute to restoration of age-related mitochondria function and metabolic activity in older animals. This contributes to improvements in energy, general health, mental acuity, immune system function, fur appearance and muscle mass.

Carnitine is available in many forms and all those are included in the invention of the combination of carnitine and thioctic acid. Carnitine and carnitine derivatives have been used as metabolites in animal husbandry and for human diet and therapy. U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); U.S. Pat. No. 5,240,961 (Method of treating reduced insulin-like growth factor and bone loss associated with aging); etc. Most preferably, the carnitine is acetyl-L-carnitine.

A daily dosage of carnitine is about 1 mg to 6 g. Preferably the daily dose of carnitine is 10-1,000 mg. More preferably, the daily dose of carnitine is about 30-500 mg. More preferably, the daily dose of carnitine is at least about 10 milligrams (0.01 g) per day. The daily dose of carnitine also depends on the weight of the pet, which varies widely for adult breeds of dogs and cats. A preferred weight-based daily dosage of carnitine for mature pets is 0.5 to 100 mg/kg/day.

By lipoic acid or thioctic acid is meant a mitochondrially active antioxidant which physiologically comprises a metabolically reactive thiol group. Mitochondrially active antioxidants including vitamins (especially C, E, B and D), glutathione, N-acetyl cysteine (NAC), lipoic acid, their derivatives, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); U.S. Pat. No. 5,326,699 (Serum-free medium for culturing animal cells); etc. Preferably, the compound is at least one of glutathione, N-acetyl cysteine and lipoic acid. Most preferably, the compound is the R-enantiomeric form of lipoic acid. Metabolites of lipoic acid have been found to have a longer half life and also are suitable for supplementation.

A daily dosage of lipoic acid is about 5 mg to 8 g. Preferably the daily dose of lipoic acid is 10-1,000 mg. More preferably, the daily dose of lipoic acid is about 30-700 mg. Most preferably, the daily dose of lipoic acid is at least about 40 milligrams (0.04 g) per day. The daily dose of lipoic acid also depends on the weight of the pet, which varies widely for adult breeds of dogs and cats. A preferred weight-based daily dosage of lipoic acid for mature pets is 0.15 to 50 mg/kg/day.

Q10 is an important supplement. In groups of males and females ranging from 90-106 years, the prevalence of inadequate Q10 status was 40% for women and 24% for men. In women, the decreased Q10 was associated with impaired natural killer cell effectiveness (p<0.05), indicating decreased ability to fight infections and quickly eliminate individual cancer cells as they first develop. Q10 also appears to block programmed cell death, or apoptosis, through its action in the mitochondria (Kagan T et al, Ann NY Acad Sci 887:31-47, 1999). Furthermore, Q10 in its reduced from of ubiquinol-10 which is normally present in the blood, appears to protect human lymphocytes from oxidative damage to DNA (Tomasetti et al, Free Radic Biol Med 27 (9-10):1027-32, November 1999). No important adverse effects have been reported from experiments using daily supplements of up to 200 mg Q10 for 6-12 months and 100 mg daily for up to 6 y. Overvad K et al. Eur J Clin Nutr 53(10):764-70, 1999.

Q10 also may contribute to anti-aging effect by protecting against atherosclerosis which also results from oxidative stress. Pedersen H S, et al. Biofactors 9(2-4): 319-23, 1999). Q10 also improves the tolerance of the senescent myocardium to aerobic and ischemic stress in human atrial tissue and rats. Q10 corrected the age-specific diminished recovery of function in older hearts so that older hearts recovered function at a similar rate to younger ones (Rosenfeldt F L et al. Biofactors 9(2-4): 291-9, 1999).

As for the supplemental dose of Q10, older Finnish men obtained benefit from 100 mg/day. A woman deficient in Q10 received 150 mg/kg and rapidly improved (Sobriera et al. Neurology 48:1283-43, 1997). Q10 has also been used at dose of about 200 mg/day to help improve heart function in persons with hypertrophic cardiomyopathy. Based on this information, a supplemental dosage for pets ranges from about 0.1 mg/day to about 100 mg/day. The daily dose of coenzyme Q also depends on the weight of the pet, which varies widely for adult breeds of dogs and cats. A preferred weight-based daily dosage of lipoic acid for mature pets is 0.015 to 20 mg/kg/day.

Because creatine intake is often decreased in older individuals, creatine supplementation should be considered. Many athletes have taken doses of creatine up to 75 grams a day for years without known adverse effects, aside from weight gain, often attributed to increased muscle mass. Creatine may be most beneficial when ingested with glucose, which tends to increase creatine absorption. Often athletes ingest loading doses of 20 g/day divided into four doses for 5 days to one week. Then they take a maintenance dose of 5 g/day. Benefit in one week in older individuals (40-73) has also been seen from a 20 g/day dose, in the form of increased skeletal muscle strength and endurance. It has been reported that 1.5 g-25 g/day are safe for a period of at least a year. A suitable dosage range for pets is about 0.15 g/day to 25 g/day, preferably 0.3-2.5 grams per day and most preferably about 0.5 g/day. On a weight basis, the mature pet would benefit from 15 mg to 1000 mg/kg/day. Creatine is available as a salt, monohydrate, phosphate and citrate.

In addition to the compositions mentioned above and the examples given below, animal snacks, “treats”, and supplements also benefit from the addition of a carnitine and a form of thioctic acid. The carnitine, thioctic acid, and optionally coenzyme Q and/or creatine can be added to bulk powders or dried or canned pet food. The combination of carnitine, thioctic acid, and optionally coenzyme Q and/or creatine can be mixed with any cooked or uncooked food.

The combination of carnitine, thioctic acid, and optionally coenzyme Q and/or creatine is provided in pet formulations, dried or canned or as a supplement for addition thereto. Animals expected to benefit from the composition include, but are not limited to, dogs, cats, horses, birds and fish.

The formulations and/or content of these products are on the product label or are otherwise publicly available.

Additional nutrients are important in older animals, including calcium, vitamin D, Vitamins B12, folic acid, B6, niacin, vitamins C or E, iron and zinc. Many of these nutrients have been found to be deficient in the diets of elders and should be appropriately supplemented along with carnitine, thioctic acid, and optionally coenzyme Q and/or creatine.

The inventive combination(s) also are conveniently provided in pill or capsule form. A preferred formulation provides lipoic acid and carnitine, optionally in combination with coenzyme Q10 and or creatine, in a timed release formulation to provide a steady supply of the nutrients to the mitochondria which work 24 hours a day. One method of accomplishing timed release is chemically combining the micronutrient(s) with other molecules, which generally slows the process of making the micronutrient(s) available. Also the use of different salts of the micronutrients with different dissolution rates provides for gradual and appropriate release of the product.

Besides these methods, two other basic systems are used to control release for oral administration: coating a core comprising the micronutrient(s) and excipients (coated system) and incorporating the micronutrient(s) into a matrix (matrix system). Coated systems involve the preparation of product-loaded cores and coating the cores with release rate-retarding materials. Product-loaded cores can be formulated as microspheres, granules, pellets or core tablets. There are many known core preparation methods, including, but not limited to, 1) producing granules by top spray fluidized bed granulation, or by solution/suspension/powdering layering by Wurster coating, 2) producing spherical granules or pellets by extrusion-spheronization, rotary processing, and melt pelletization; 3) producing core tablets by compression and coating with a release rate-retarding material; 4) producing microspheres by emulsification and spray-drying.

Matrix systems embed the micronutrient in a slowly disintegrating or non-disintegrating matrix. Rate of release is controlled by the erosion of the matrix and/or by the diffusion of the micronutrient(s) through the matrix. In general, the active product substance, excipients and the release rate-retarding materials are mixed and then processed into matrix pellets or tablets. Matrix pellets can be formed by granulation, spheronization using cellulosic materials, or by melt pelletization using release retardant materials, while matrix tablets are prepared by compression in a tablet press. An example of a cellulosic material is hydroxypropylmethyl-cellulose as the release rate retarding material.

Coated or matrix pellets can be filled into capsules or compression tabletted. The rate of release can be further modified by blending coated or matrix pellets with different release rates of the same product to obtain the desired product release profile. Pellets containing any of lipoic acid, carnitine, coenzyme Q10 or creatine can be blended to form a combination product.

Convenient assays for the requisite bioactivities are described above or in the references cited herein. For example, cardiolipin content is readily assayed as referenced in Guan, Z. Z., Soderberg, M., Sindelar, P., and Edlund, C. Content and Fatty Acid Composition of Cardiolipin in the Brain of Patients with Alzheimer's Disease. Neurochem. Int. 25: 295-300, 1994 and oxidant production (DCFH) may be assayed as described by LeBel, C. P., Ischiropoulos, H., and Bondy, S. C. Evaluation of the Probe 2′,7′-Dichlorofluorescin as an Indicator of Reactive Oxygen Species Formation and Oxidative Stress. Chem. Res. Toxicol. 5: 227-231, 1992. Assays for parameters of aging such as host activity and behavior such as grooming, sexual activity, dominance, coat condition, wound repair, including molecular lesions, muscle strength and tone, kidney appearance and function, etc. are similarly well known in the art.

### EXAMPLE 1

The Eukanuba Senior Maintenance (IAMS) is formulated to help nutritionally stabilize the senior dog's digestive system. The senior maintenance diet was formulated with 50% more antioxidants than their adult formulas, from sources such as vitamin E and Beta-Carotene. This is intended for small breeds over 8 years of age, medium breeds over 7 years of age, large breeds over 6 years of age, and giant breeds over 5 years of age. Its ingredients include chicken by-product meal, corn meal, ground grain sorghum, ground whole grain barley, chicken, fish meal, dried beet pulp (sugar removed), chicken fat (preserved with mixed tocopherols, a source of vitamin E, and citric acid), dried egg product, brewers dried yeast, vitamins and minerals. It provides 4,219 kcal/kg or 350 kcal/cup in the following distribution: protein 27%, fat 28%, and carbohydrate 45%.

Guaranteed Analysis:

| Nutrient | (percent) |
|---|---|
| Crude Protein min. | 26.0% |
| Crude Fat min. | 10.0% |
| Crude Fiber max. | 4.0% |
| Moisture max. | 10.0% |
| Omega-6 Fatty Acids min. | 1.75% |
| Omega-3 Fatty Acids min. | 0.25% |

To improve the nutritional value for senior dogs, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 2

The Iams Senior Formula for Cats meets most of the special nutritional needs of cats over 7 years of age. Iams Senior Formula provides essential protein levels. And with 30% less fat than Iams Original Formula, the cat avoids excess weight gain. Ingredients include chicken by-product meal, chicken, rice flour, corn meal, dried beet pulp (sugar removed), dried egg product, natural chicken flavor, fish meal, potassium chloride, brewers dried yeast, dl-methionine, calcium carbonate, salt, choline chloride, vitamin E supplement, zinc oxide, chicken fat (preserved with mixed tocopherols, a source of vitamin E, and citric acid), vitamins and minerals. This formulation provides 4,108 kcal/kg, or 373 kcal/cup from the following sources: protein 32%, fat 34%, and carbohydrate 34%.

Guaranteed Analysis:

| Nutrient | (percent) |
|---|---|
| Crude Protein min | 32.0% |
| Crude Fat min | 14.0% |
| Crude Fat max | 16.5% |
| Crude Fiber max | 3.0% |
| Moisture max | 10.0% |
| Ash max | 6.75% |
| Magnesium max | 0.099% |
| Taurine min | 0.15% |
| Vitamin B not less than | 200 IU/kg |

To properly supply nutrients to older cats, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 3

Kasco® Maintenance Dog Food is a low-protein, low-fat, low-calorie formula for maintenance for less active and older dogs. It contains the following ingredients: Ground yellow corn, poultry by-product meal, poultry fat (preserved with mixed tocopherols), beet pulp, salt, calcium carbonate, dicalcium phosphate, choline chloride, zinc proteinate, vitamin E supplement, ascorbic acid, zinc oxide, manganese proteinate, copper proteinate, extract of rosemary, manganous oxide, copper sulfate, vitamin A acetate, niacin supplement, calcium pantothenate, vitamin B12 supplement, vitamin D3 supplement, pyridoxine hydrochloride, riboflavin supplement, thiamine mononitrate, calcium iodate, biotin, sodium selenite, folic acid. Its guaranteed analysis is crude protein (min) 22%, crude fat (min) 10%, crude fiber (max) 3.5%, and moisture (max) 11%.

To formulate this product specifically for older dogs, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 4

Heinz provides a blend of ingredients called the Custom Fitness™ formula of Cycle Senior. It contains rice and oatmeal; vitamins A, C, and E plus beta-carotene; and no added salt; and limited calcium, phosphorus, and fat. Ingredients of the dry formula include corn, soybean hulls, chicken by-product meal, feeding oat meal, brewers rice, whole wheat, animal fat (BHA used as a preservative), animal digest, condensed grain fermentation solubles, bone phosphate, calcium carbonate, potassium chloride, L-lysine monohydrochloride, L-threonine, D,L-methionine, choline chloride, minerals (ferrous sulfate, zinc oxide, manganous oxide, copper sulfate, calcium iodate, sodium selenite), vitamins (vitamin E supplement, niacin, D-calcium pantothenate, riboflavin supplement, pyridoxine hydrochloride, thiamin mononitrate, vitamin A supplement, folic acid, biotin, vitamin B12 supplement, vitamin D3 supplement), antioxidant blend (ascorbic acid, beta carotene, marigold extract), BHA (preservative), tocopherols (preservative), citric acid (preservative), rosemary extract (preservative).

Dry Formula Guaranteed Analysis

| | As Fed | Dry Weight |
|---|---|---|
| Protein | 19.50% | 21.31% |
| Sodium | 0.08% | 0.09% |
| Crude Fat | Not less than 9.00% | |
| Crude Fiber | Not more than 4.00% | |
| Moisture | Not more than 12.00% | |
| Calcium | Not less than 0.60% | |
| Phosphorous | Not less than 0.50% | |
| Sodium | Not more than 0.15% | |
| Calories per cup | 350 Calories | |

The canned formula has the following ingredients: water sufficient for processing, chicken, wheat flour, oatmeal, turkey, wheat gluten, brewer's rice, meat by-products, soybean oil, guar gum, vitamins (vitamin E supplement, niacin, D-calcium pantothenate, riboflavin supplement, pyridoxine hydrochloride, thiamin mononitrate, vitamin A supplement, folic acid, biotin, vitamin B12 supplement, vitamin D3 supplement), sodium tripolyphosphate, potassium chloride, titanium dioxide, calcium sulfate, choline chloride, minerals (ferrous sulfate, zinc oxide, manganous oxide, copper sulfate, calcium iodate, sodium selenite), DL-methionine, carrageenan, FD&C yellow 6, iron oxide, antioxidant blend (ascorbic acid, beta carotene, marigold extract), and FD&C yellow 5.

Canned Formula Guaranteed Analysis

| | As Fed | Dry Weight |
|---|---|---|
| Protein | 5.13% | 26.50% |
| Sodium | 0.15% | 0.77% |
| Crude Fat | Not less than 3.0% | |
| Crude Fiber | Not more than 5.0% | |
| Moisture | Not more than 82.0% | |
| Calories per cup | 288 Calories | |

To convert these products to support the metabolism of active seniors, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 5

Mera Dog Sensitive is a maintenance formula suitable for the senior and less active dog. Main ingredients are turkey and rice, which are highly digestible and provide protein with reduced calories. Specifically, ingredients include rice (60%), turkey meat meal (20%), poultry fat, beet fiber, linseed, brewers yeast, minerals, dehydrated egg, poultry meat hydrolysate, DL-methionine, L-lysine. Additives include vitamin A 15,000 I.E./kg, vitamin D3 1.500 I.E./kg, vitamin E 120 mg/kg, and copper 19 mg/kg. Guaranteed Analysis is crude protein 21.0%, crude fat 9.0%, crude fiber 2.5%, crude ash 7.0%, calcium 1.2%, phosphorus 1.0%, and sodium 0.4%.

To convert this product to support the metabolism of active seniors, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 6

NuFood was created for pet owners concerned about giving their dogs top quality nutrition. NuFood is made with 100% pure chicken meat (no by-products) and is prepared to provide a pure, tasty and highly digestible meal. There are no gels, dyes or artificial flavors; and all ingredients are approved for human consumption. The main ingredients are chicken meat, corn, breadcrumbs, and water. Ingredients include 100% pure chicken meat, corn gluten meal, toasted wheat crumbs, propylene glycol, corn syrup solids, wheat flour, corn flour, glucono delta lactone, salt, citric acid, sodium nitrite, seasoning, and water sufficient for processing.

To convert this product to support the metabolism of active seniors, the following ingredients are added: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 7

The Science Diet Canine Senior product described in the Background can benefit from the following additional ingredients: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 8

The Canine Senior formula described in the Background can benefit from the following additional ingredients α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

### EXAMPLE 9

The Science Diet Feline Senior canned fish formula (as well as other Science Diet cat foods) can benefit from the following additional ingredients: α-lipoic acid at about 0.15 to 50 mg/kg body weight/day, carnitine at about 0.5 to 100 mg/kg/day, and optionally Q10 at about 0.01 mg/kg/day and/or creatine at about 15 mg to about 1 g/kg/day.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A pet food formulated for senior pets, the food so formulated as to provide the following chemicals in a daily dosage comprising
a) α-lipoic acid at about 0.15 to 50 mg/kg body weight/day,
b) carnitine at about 0.5 to 100 mg/kg/day, and
c) optionally coenzyme Q at about 0.01 mg/kg/day and/or creatine at about 15 mg to about 1 g/kg/day.

2. The pet food of claim 1 wherein the carnitine is acetyl-L-carnitine.

3. The pet food of claim 1 wherein the α-lipoic acid is R-α-lipoic acid.

4. The pet food of claim 1, wherein coenzyme Q is coenzyme Q10.

5. A pet treat for senior pets, the treat formulated so as to provide
a) α-lipoic acid at about 0.15 to 50 mg/kg body weight/day,
b) carnitine at about 0.5 to 100 mg/kg/day, and
c) optionally coenzyme Q at about 0.01 mg/kg/day and/or creatine at about 15 mg to about 1 g/kg/day.

6. The pet treat of claim 5, wherein the carnitine is acetyl-L-carnitine.

7. The pet treat of claim 5, in which the α-lipoic acid is R-α-lipoic acid.

8. The pet treat of claim 5, wherein coenzyme Q is coenzyme Q10.

9. A pet supplement for mature pets, the supplement offering
a) α-lipoic acid at about 0.15 to 50 mg/kg body weight/day,
b) carnitine at about 0.5 to 100 mg/kg/day, and
c) optionally coenzyme Q at about 0.01 mg/kg/day and/or creatine at about 15 mg to about 1 g/kg/day.

10. The supplement of claim 9 wherein the carnitine is acetyl-L-carnitine.

11. The supplement of claim 9 wherein the α-lipoic acid is R-α-lipoic acid.

12. The supplement of claim 9, wherein the coenzyme Q is coenzyme Q10.
